(19) United States
(12) Patent Application Publication
(10) Pub. No.: US 2010/0034783 A1
(43) Pub. Date: Feb. 11, 2010

(54) Medical kit and using method thereof

(76) Inventors: Hyun-Mi Son, Seoul (KR); Cheong-Ho Chang, Seoul (KR); Jae-Deog Jang, Seoul (KR)

Correspondence Address: GWIPS;Peter T. Kwon, Gwacheon P.O. Box 72, 119 Byeolyang Ro, Gwacheon City, Gyeonggi-Do, 427-600, KR

(21) Appl. No.: 12/311,783

(22) PCT Filed: Nov. 3, 2006
(86) PCT No.: PCT/KR2006/004549

§ 371 Date: Apr. 13, 2009

(30) Foreign Application Priority Data
Oct. 27, 2006 (KR) 10-2006-0105127

(51) Int. Cl. A61K 35/12; A61B 10/00; B65D 81/18; B65B 7/28; A61B 5/15; C12N 5/02; A01N 1/02

(52) U.S. Cl. 424/93.7; 600/562; 62/64; 53/468; 600/573; 435/378; 435/2

(57) ABSTRACT

An aseptic/sterile medical kits are comprising a cartilage regeneration kit, a bone regeneration kit or an umbilical cord blood storage kit in a configuration that each process performs according to functionally-specialized kit sets for each step, via division of overall processes into corresponding steps for isolation, culture, collection and storage of cells, and implantation of desired cells into target sites of the body. The cartilage is regenerated by cartilage tissue collection; chondrocyte isolation; chondrocyte medium change and subculture; preparation of chondrocyte therapy product; media for isolation/culture/preparation/cryopreservation of cells; and media for isolation/culture/cryopreservation of cells, using the cartilage regeneration kit. The bone is regenerated by bone marrow collection; osteoblast isolation; osteoblast medium change and subculture; and preparation of osteoblast therapy product, using the bone regeneration kit. Additionally, the umbilical cord blood is stored by umbilical cord blood collection; hematopoietic stem cell isolation; and cryopreservation of hematopoietic stem cells, using the umbilical cord blood storage kit.

### TECHNICAL FIELD

The present invention relates to a medical kit and a method of using the same. More specifically, the present invention relates to a method of using a kit associated with the preparation and implantation of a cell therapy product intended for regeneration of cartilage and bone and storage of umbilical cord blood, and the treatment of umbilical cord blood. In particular, the present invention enables fabrication and supply of various materials, culture media and solutions, necessary for isolation, culture, collection, implantation and storage processes of desired cells, in the form of a single kit for easy and convenient use. Hence, the present invention can realize reduction of problems such as high production costs, a long period of production time and high labor costs in the preparation of cell therapy products and the storage of umbilical cord blood, suffered by conventional arts. Therefore, the present invention accomplishes remarkably improved quality and reliability of the products and thereby is very useful to enhance customer satisfaction.

### BACKGROUND ART

As is well known in the art, there is now great and increasing interest in tissue regeneration, due to the severe shortage of donor organs for transplantation. However, it is very difficult to develop cell therapy products as an alternative to the tissue regeneration, and further it is not easy to establish a storage method of umbilical cord blood. In particular, the cell therapy products are not therapeutic measures which are widely commercially available in the current medical field. Further, there is no case of fabrication of aseptic/sterile medical appliances and culture media into a kit in the form of a single set, such that the cell therapy products can be easily and conveniently prepared without difficulty and then implanted to target areas.

Further, the tissue engineering is an area of biotechnology aimed at providing normal biological functions of organs or tissues, through restoration, reconstruction, regeneration or replacement of damaged tissues or organs. Although the tissue engineering has been previously unfamiliar to either biologists or engineers until only few years ago, it has now become a well-known and rapidly growing branch of science. The tissue engineering has become a noticeable area, as it was named as ‘one of the most promising research fields of the 20th century’, as selected by Time Magazine (May 2005). This is believed to be due to the product of consistent and indefatigable efforts of experts who are engaged in a variety of fields. Also in Korea, the tissue engineering is now rapidly growing with drawing a great deal of interest.

At the early stage of the tissue engineering, research was largely intent on in vitro fabrication of tissues and organs which are anatomically or histologically similar to human tissues and organs. Thereafter, the tissue engineering has placed great importance on the functions of the thus-formed tissues. Through consistent and intensive study and research, many efforts have been made on clinical application of the tissue engineering products and technologies throughout various areas, starting from artificial skin. Recently, tissue regeneration is also addressed in conjunction with tissue reconstruction.

One of the fundamental constituent elements of the tissue engineering is a cell. For example, allogeneic, xenogeneic or autologous cells have been used in study of the tissue engineering. In order to avoid immunological rejection which may occur upon implantation of transplants, the allogeneic or xenogeneic cells are allowed to induce secretion of substances necessary for the human body, thereby aiming at improving biological functions of in vivo tissues. However, the use of the allogeneic or xenogeneic cells suffers from limitations of practical applications, which has led to the preference for use of autologous cells without causing immune rejection. A great deal of study has been made using autologous cells. Currently, practical clinical trials after completion of animal tests in a variety of areas are being actively undertaken with reporting of some satisfactory results. Unlike artificial biomaterials, injection therapies utilizing such autologous cells are preferred due to no risk of foreign body reactions. Formation of tissues via the injection therapy has been already clinically attempted in various kinds of tissues. In particular, it can be said that introduction and assistance of an endoscope have led to expansion of application range thereof.

However, the overall process for preparation of the cell therapy products utilizing these autologous cells is somewhat complicated and therefore cannot be easily carried out. Upon handling of various plastic wares, solutions and media used in the preparation of the cell therapy products, cells to be implanted into the defect area of the body may be contaminated. Further, patients may miss an opportunity of timely implantation as a period of the cell culture time is prolonged.

### DISCLOSURE

### Technical Problem

Therefore, the present invention has been made in view of the problems associated with conventional art as discussed above, and it is a first object of the present invention to provide a method of using a kit associated with the preparation and implantation of a cell therapy product intended for regeneration of cartilage and bone and storage of umbilical cord blood, and the treatment of the umbilical cord blood. In particular, the present invention enables construction and supply of various materials, culture media and solutions, necessary for isolation, culture, collection, implantation and storage processes of desired cells, in the form of a single kit for easy and convenient use. Hence, the present invention can realize reduction of problems such as high production costs, a long period of production time and high labor costs in the preparation of cell therapy products and the storage of umbilical cord blood, suffered by conventional arts.

A second object of the present invention is to provide an aseptic and sterile medical kit which enables safe isolation, culture, collection and storage of cells from the collected cartilage, bone marrow tissues and umbilical cord blood, and implantation of the desired cells into the target sites of the body. For this purpose, the medical kit is configured in the form of a single kit set, such that each process can be performed according to the functionally-specialized kit sets for corresponding steps, via division of the overall process into the corresponding steps.

A third object of the present invention is to achieve standardization of all processes, associated with the preparation of a cell therapy product intended for regeneration of cartilage and bone, according to the corresponding steps, and fabrication of a kit suited for the standardized processes. Therefore, the present invention enables easy and convenient use of the kit at a time, which leads to activation and promotion of production of the cell therapy products and further, commercialization of a novel therapy using the tissue engineering.

A fourth object of the present invention is to provide standardization of a protocol for storage of umbilical cord blood, which has the same configuration as illustrated in the above-mentioned kit for cartilage and bone regeneration, fabrication of functionally-specialized kits corresponding to each process, and convenient use thereof at a time. Therefore, as compared to conventional storage/treatment methods, reduction of costs, time and manpower associated with the storage of the umbilical cord blood can be achieved.

A fifth object of the present invention is to provide a medical kit and a method of using the same, which are suited for enhancing customer satisfaction via remarkably improved quality and reliability of the products.

### Technical Solution

In accordance with an aspect of the present invention, the above and other objects can be accomplished by the provision of an aseptic/sterile medical kit for cartilage regeneration comprising a cartilage regeneration kit in a configuration that each process can be performed according to the functionally-specialized kit sets for each step, via division of the overall processes into the corresponding steps for isolation, culture, collection and storage of cells, and implantation of the desired cells into the target sites of the body. The cartilage regeneration kit includes a cartilage tissue collection kit, a chondrocyte isolation kit, a chondrocyte medium change and subculture kit, and a chondrocyte therapy product preparation kit. Therefore, the cartilage is regenerated by cartilage tissue collection; chondrocyte isolation; chondrocyte medium change and subculture; preparation of a chondrocyte therapy product; media for isolation/culture/preparation/cryopreservation of cells; and media for isolation/culture/cryopreservation of cells, using the cartilage regeneration kit.

In accordance with another aspect of the present invention, there is provided an aseptic/sterile medical kit for bone regeneration comprising a bone regeneration kit in a configuration that each process can be performed according to the functionally-specialized kit sets for each step, via division of the overall processes into the corresponding steps for isolation, culture, collection and storage of cells, and transplantation of the desired cells into the target sites of the body. The bone regeneration kit includes a bone marrow collection kit, an osteoblast isolation kit, an osteoblast medium change and subculture kit, and an osteoblast therapy product preparation kit. Therefore, the bone is regenerated by bone marrow collection; osteoblast isolation; osteoblast medium change and subculture; and preparation of an osteoblast therapy product, using the bone regeneration kit.

In accordance with yet another aspect of the present invention, there is provided an aseptic/sterile medical kit for storage of hematopoietic stem cell comprising an umbilical cord blood storage kit in a configuration that each process can be performed according to the functionally-specialized kit sets for each step, via division of the overall processes into the corresponding steps for isolation, culture, collection and storage of cells, and implantation of the desired cells into the target sites of the body. The umbilical cord blood storage kit includes an umbilical cord blood collection kit, a hematopoietic stem cell isolation kit, and a hematopoietic stem cell cryopreservation kit. Therefore, the umbilical cord blood is stored by umbilical cord blood collection; hematopoietic stem cell isolation; and cryopreservation of hematopoietic stem cells, using the umbilical cord blood storage kit.

### DESCRIPTION OF THE DRAWINGS

The above and other objects, features and other advantages of the present invention will be more clearly understood from the following detailed description taken in conjunction with the accompanying drawings, in which:

FIG. 1 is a schematic view showing a configuration of a cartilage regeneration kit which is applied to the present invention;

FIG. 2 is a schematic view showing a configuration of a bone regeneration kit which is applied to the present invention;

FIG. 3 is a schematic view showing a configuration of an umbilical cord blood storage kit which is applied to the present invention;

FIG. 4 is a photograph of a cartilage tissue collection kit which is applied to the present invention;

FIG. 5 is a photograph of a chondrocyte isolation kit which is applied to the present invention;

FIG. 6 is a photograph of CRM-P1 Medium Change Kit which is applied to a chondrocyte medium change kit of the present invention;

FIG. 7 is a photograph of CRM-P2 Medium Change Kit which is applied to a chondrocyte medium change kit of the present invention;

FIG. 8 is a photograph of CRM-P3 Medium Change Kit which is applied to a chondrocyte medium change kit of the present invention;

FIG. 9 is a photograph of CRM-P1 Subculture Kit which is applied to a chondrocyte subculture kit of the present invention;

FIG. 10 is a photograph of CRM-P2 Subculture Kit which is applied to a chondrocyte subculture kit of the present invention;

FIG. 11 is a photograph of CRM-Collection Kit which is applied to a cell collection kit in a chondrocyte therapy product preparation kit of the present invention;

FIG. 12 is a photograph of CRM-Delivery Kit which is applied to a delivery kit in a chondrocyte therapy product preparation kit of the present invention;

FIG. 13 is a photograph of CRM-Implantation Kit which is applied to an implantation kit in a chondrocyte therapy product preparation kit of the present invention;

FIG. 14 is a photograph of a medium kit for isolation/culture/preparation/cryopreservation of cells which is applied to the present invention;

FIG. 15 is a photograph of a medium kit for isolation/culture/cryopreservation of cells which is applied to the present invention;

FIG. 16 is a photograph of an umbilical cord blood collection kit which is applied to the present invention;

FIG. 17 is a photograph a hematopoietic stem cell isolation kit which is applied to the present invention; and

FIG. 18 is a photograph a medium kit for isolation/cryopreservation of hematopoietic stem cells which is applied to the present invention.

### BEST MODE

Hereinafter, the preferred embodiments of the present invention for accomplishing the above-mentioned objects will be described in more detail with reference to the accompanying drawings.

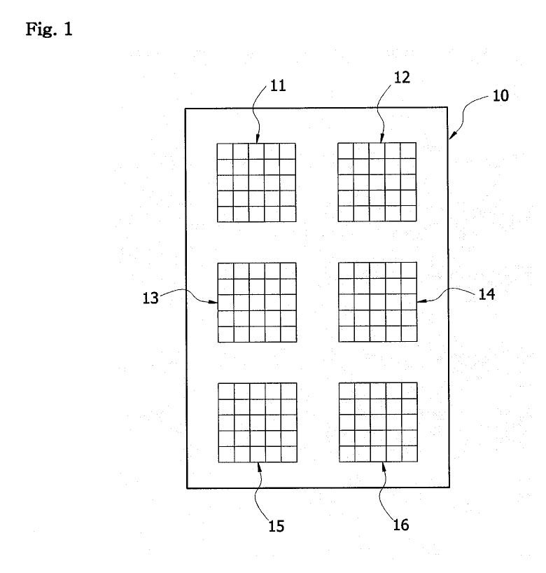

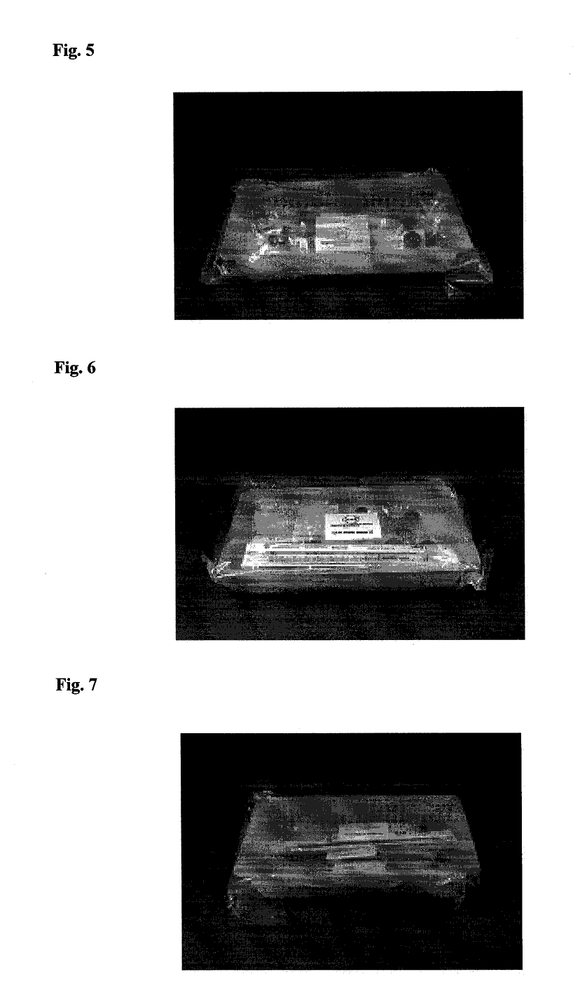

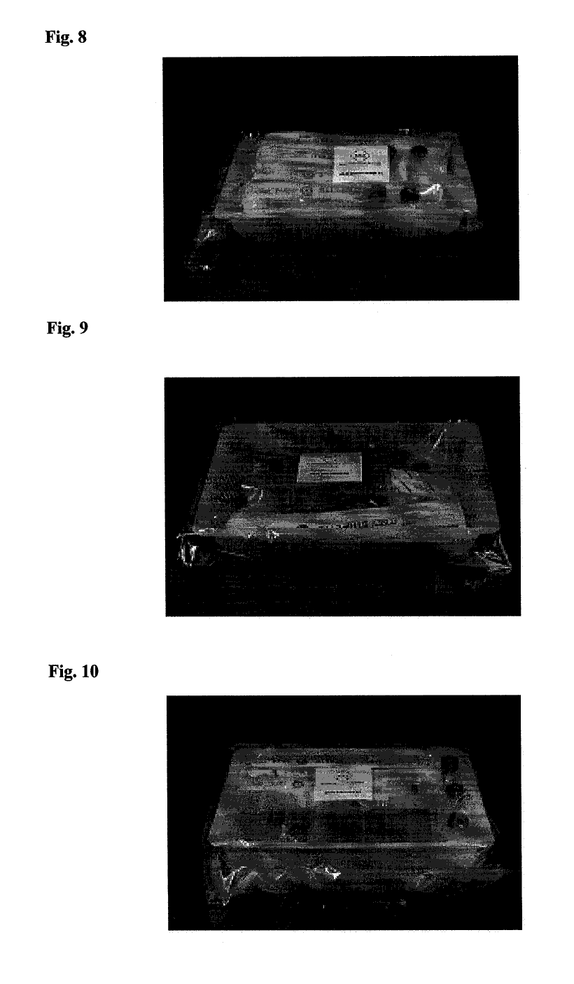

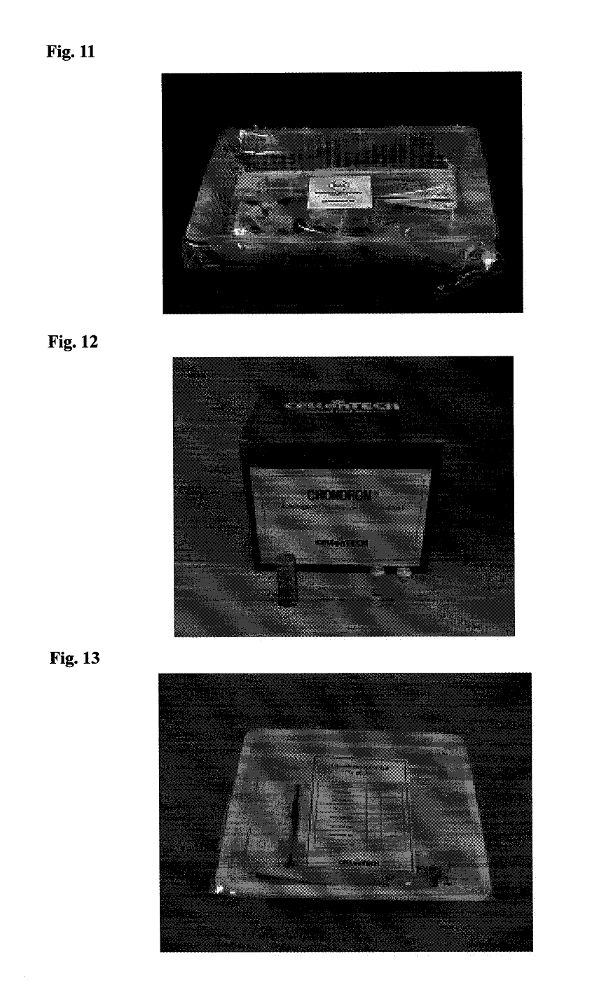

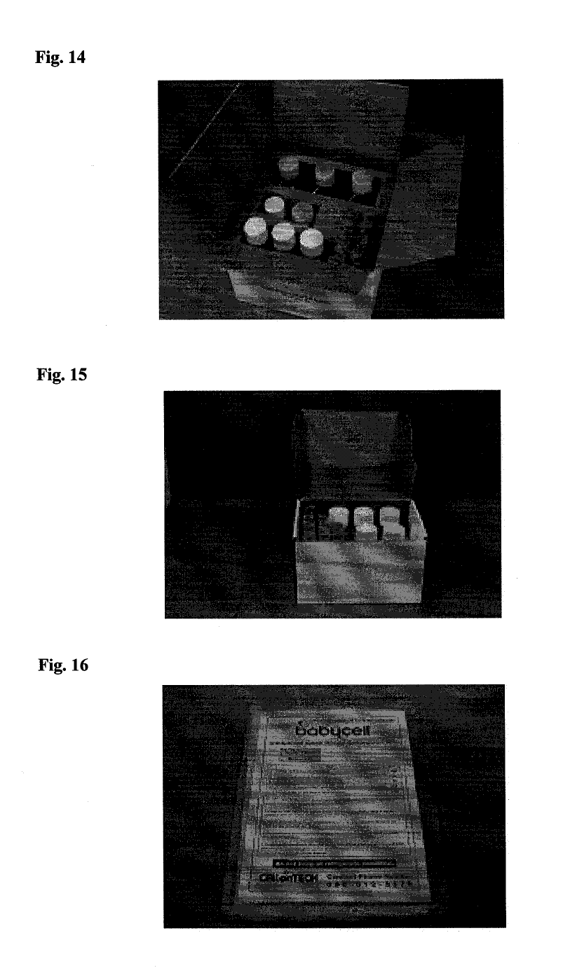

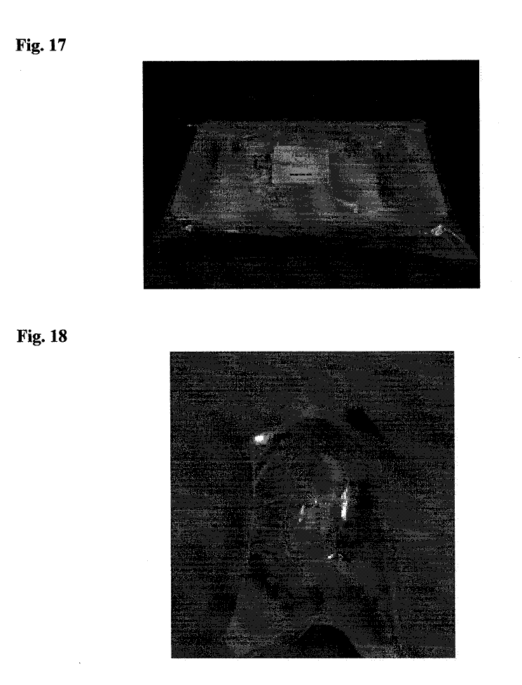

A medical kit and a method using the same, which are applied to the present invention, are constituted as shown in FIGS. 1 through 18.

In connection with description of the present invention hereinafter, if it is considered that description of known functions or constructions related to the present invention may make the subject matter of the present invention unclear, the detailed description thereof will be omitted.

Terms which will be described hereinafter are established taking into consideration functions in the present invention and may vary according to manufacturer's intention or a usual practice in the related art. Therefore, the terms used herein should be defined based on the contents of the specification of the present invention.

First, the technological constitution of the present invention is composed broadly of a kit intended for cartilage regeneration, a kit intended for bone regeneration, and a kit intended for storage of umbilical cord blood.

That is, the kit 10 for cartilage regeneration is a plasticware kit, and comprises:

1. A cartilage tissue collection kit (CRM-Transport Kit),

2. A chondrocyte isolation kit (CRM-Initiation Kit [CRM-Isolation Kit, CRM-Primary Kit]),

3. A chondrocyte medium change and subculture kit (CRM-Processing Kit [CRM-P1 Subculture Kit, CRM-P2 Subculture Kit, CRM-P1 Medium Change Kit, CRM-P2 Medium Change Kit, CRM-P3 Medium Change Kit]), and

4. A chondrocyte therapy product preparation kit (CRM-Product Kit [CRM-Collection Kit, CRM-Delivery Kit, CRM-Medium Packaging Kit, CRM-Implantation Kit]), which are composed of a variety of sub-kits, respectively.

The cartilage regeneration kit further includes a medium kit for isolation/culture/preparation/cryopreservation of cells (CRM-RF Kit [CSB-I, CSB-II, CSM, CPM, CCM, CCB]) and a medium kit for isolation/culture/cryopreservation of cells (CRM-FZ Kit [Cartisepor, STA, PCF, Pass solution, Cell stock solution, CSM AD solution, CPM AD solution, Neutro solution]), which assist in isolation, culture, collection and implantation of cells.

In addition, the kit 20 for bone regeneration is a plasticware kit, and includes:

1. A bone marrow collection kit (BRM-Transport Kit),

2. An osteoblast isolation kit (BRM-Initiation Kit [BRM-Isolation Kit, BRM-Primary Kit]),

3. An osteoblast medium change and subculture kit (BRM-Processing Kit [BRM-P1 Subculture Kit, BRM-P2 Subculture Kit, BRM-P1 4. Medium Change Kit, BRM-P2 Medium Change Kit, BRM-P3 Medium Change Kit]),

4. An osteoblast therapy product preparation kit (BRM-Product Kit [BRM-Collection Kit, BRM-Delivery Kit, BRM-Medium Packaging Kit, BRM-Implantation Kit]),

5. A medium kit for isolation/culture/preparation/cryopreservation of osteoblasts (BRM-RF Kit [OSB, BSB, BSM, BPM, BCM, BCB, BDB, BNB]), and

6. A medium kit for isolation/culture/cryopreservation of osteoblasts (BRM-FZ Kit [STA, PCF, Pass solution, Cell stock solution, OSM AD solution, OPM AD solution, Neutro solution]), in the form of a single set.

Finally, the kit 30 for storage of umbilical cord blood is composed of three kits:

1. An umbilical cord blood collection kit (USC RM-Transport Kit),

2. A hematopoietic stem cell isolation kit (USC RM-Processing Kit), and

3. A hematopoietic stem cell cryopreservation kit (USC RM-Storage Kit), and further includes a medium for isolation and cryopreservation of hematopoietic stem cells (USC RM-RF Kit) and a medium for isolation and cryopreservation of hematopoietic stem cells (USC RM-FZ Kit).

Hereinafter, the present invention will be described in more detail.

As shown in FIG. 1, an aseptic/sterile kit for cartilage regeneration is provided according to the present invention. The kit includes a cartilage regeneration kit 10 in a configuration that each process can be performed according to the functionally-specialized kit sets for each step, via division of the overall processes into the corresponding steps for isolation, culture, collection and storage of cells, and implantation of the desired cells into the target sites of the body. The cartilage regeneration kit 10 includes a cartilage tissue collection kit 11, a chondrocyte isolation kit 12, a chondrocyte medium change and subculture kit 13, and a chondrocyte therapy product preparation kit 14.

Herein, the cartilage tissue collection kit 11 includes a hard tissue collection kit for collection of cartilage tissues, a Universal container which is an internal container for tissue delivery, a transport bottle which is an external container for tissue delivery, a blue tip (1 mL) used to take 1 mL of a solution, a styrofoam box which is an internal box for tissue delivery under refrigeration conditions, sponge used for introduction of the transport bottle into the styrofoam box, and a Koolit refrigerant to keep the cold conditions.

In addition, the chondrocyte isolation kit 12 includes a Pasteur pipette for solution removal, T25 (plug seal) as a cell culture vessel with a plug-seal cap (a 25 cm² flask), a cell strainer which is placed over a 50 mL centrifuge tube and separates cells from the solution by passing the resulting suspension of cells and culture medium through it, a tube for centrifugation to wash cells, an E-tube used upon mixing trypan blue and the cell-containing medium, a blue tip to take 1 mL of a solution, a pipette for solution transfer, a syringe filter for solution filtration, a T25 cell culture flask, and a cryo tube for cryopreservation of cells.

In addition, the chondrocyte medium change and subculture kit 13, which is applied to the present invention, includes a Pasteur pipette for solution removal, a tube for centrifugation to wash cells, a pipette for solution transfer, a pipette for transfer of a large volume of a solution, a Yellow tip for taking a small volume of a solution, an E-tube used upon mixing trypan blue and the cell-containing medium, a T75 cell culture flask, and a cryo tube for cryopreservation of cells.

The chondrocyte therapy product preparation kit 14 according to the present invention includes a Pasteur pipette for solution removal, a cryo tube for cryopreservation of cells, an E-tube used upon mixing trypan blue and the cell-containing medium, a tube for centrifugation to wash cells, a pipette for solution transfer, a pipette for transfer of a large volume of a solution, a cell strainer which is placed over a 50 mL centrifuge tube and separates cells from the solution by passing the resulting suspension of cells and culture medium through it, a filter blue tip to take a solution, a rubber cap and an aluminum cap for stopping a vial, a 1 mL V-vial used to contain cells for preparation of a chondrocyte therapy product, a styrofoam box which is an internal box for delivery of a chondrocyte therapy product under refrigeration conditions, sponge used for introduction of the chondrocyte therapy product-containing vial into the styrofoam box, a Koolit refrigerant to keep cold conditions and device 2 sets for implantation of cells into a defect area.

Further, the cartilage regeneration kit 10 also includes a media kit 15 for isolation/culture/preparation/cryopreservation of cells and a media kit 16 for isolation/culture/cryopreservation of cells.

### MODE FOR INVENTION

### Examples

Now, the present invention will be described in more detail with reference to the following examples. These examples are provided only for illustrating the present invention and should not be construed as limiting the scope and spirit of the present invention.

The present invention, as constituted above, achieves regeneration of the cartilage by cartilage tissue collection; chondrocyte isolation; chondrocyte medium change and subculture; preparation of a chondrocyte therapy product; media for isolation/culture/preparation/cryopreservation of cells; and media for isolation/culture/cryopreservation of cells, using the cartilage regeneration kit 10. An example of the present invention will be described hereinafter.

### Example 1

The cartilage tissue collection process includes:

1. Unwrapping an outer paper packaging of CRM-Transport Kit contained in Cartilage RM Kit;

2. Transferring a styrofoam box with an inner packaging only to a surgical room, while leaving the removed outer packaging; and

3. Collecting cartilage tissues using a sterile hard tissue collector inside the styrofoam box, placing the collected cartilage tissues in a cartilage tissue transport container containing a red medium, double-wrapping of the cartilage tissue transport container with a styrofoam box and an outer paper box and then transferring the package into a cell culture room.

In addition, CRM-Initiation Kit of the chondrocyte isolation process includes:

1. Taking CRM-Isolation Kit out of the CRM-Initiation Kit, spraying the Kit with a sufficient amount of a 70% alcohol, and transferring the Kit to a clean bench;

2. Weighing the cartilage tissues transported on the CRM-Transport Kit, in the clean bench, using a 50 mL tube;

3. Transferring the cartilage tissues into a 50 mL tube, aliquoting CSB-I into the tube, using a 25 mL pipette, and washing the cartilage tissues;

4. Removing the cartilage tissue-washed solution by a Pasteur pipette and repeating Step 3;

5. Transferring the washed cartilage tissues into a dish and slicing the tissues into pieces using blades No. 10 and 11;

6. Transferring the sliced cartilage tissues into a 15 mL tube;

7. Aliquoting CSB-I into the tube using a 10 mL pipette;

8. Centrifuging the cartilage tissue-containing tube at 1200 rpm for 1 min;

9. Washing the tissues and removing a supernatant by a Pasteur pipette;

10. Repeating Steps 8 and 9 three times;

11. Providing two T25 flasks (plug seal), and using a 10 mL pipette, aliquoting Cartisepor into one T25 flask and aliquoting a mixture of Cartisepor and an antibiotic into the other flask;

12. Transferring ⅓ of the washed cartilage tissue into the flask containing CSB-I, and transferring the remaining ⅔ of the washed cartilage tissue into the flask containing CSB-II;

13. After transferring the tissues into the flasks, incubating them in a CO₂ incubator at 37° C., for 12 to 16 hours and stirring them at 37° C. and 100 rpm for 30 min; and

14. Placing cell strainers over 50 mL tubes, and filtering suspensions in the flasks, and

CRM-Primary Kit of the chondrocyte isolation process includes:

1. Taking a vinyl-wrapped CRM-Primary Kit out of Cartilage RM Kit, spraying the Kit with a sufficient amount of a 70% alcohol, and transferring the Kit to a clean bench;

2. Aliquoting CSM into a 50 mL tube by a 10 mL pipette;

3. Centrifuging and washing the contents of the tube, removing a supernatant by a Pasteur pipette, aliquoting CSM again into the tube, followed by washing;

4. Mixing 1/20 of cells and PCF in a cryo tube and cryopreserving the mixture; and

5. On the following day, recovering suspension cells non-adhered to the T25 flask in a 15 mL tube and inoculating the cells into the T25 flask.

In addition, CRM-P1 Medium Change Kit of the chondrocyte medium change and subculture process of the present invention includes:

1. Taking a vinyl-wrapped CRM-P1 Medium Change Kit out of Cartilage RM Kit, spraying the Kit with a sufficient amount of a 70% alcohol, and transferring the Kit to a clean bench;

2. Removing a culture medium inside a flask using a Pasteur pipette;

3. Aliquoting CSM into a 50 mL tube by a 25 mL pipette; and

4. Re-aliquoting a given amount of CSM from the aliquoted tube into the flask; and

CRM-P1 Subculture Kit of the chondrocyte medium change and subculture process includes:

1. Taking a vinyl-wrapped CRM-P1 Subculture Kit out of Cartilage RM Kit, spraying the Kit with a sufficient amount of a 70% alcohol, and transferring the Kit to a clean bench;

2. Pooling and mixing the culture medium of T25 flask in a 50 mL tube;

3. Sampling a portion of the culture medium in a 50 mL tube and an E-tube;

4. Removing the remaining culture medium by a Pasteur pipette;

5. Aliquoting CSB-II into the flask, shaking to wash the flask, and removing a supernatant by a Pasteur pipette;

6. Repeating Step 5 three times;

7. Aliquoting a Pass solution into the flask by a 10 mL pipette, and maintaining the flask in an incubator for about 3 min;

8. Aliquoting a Neutro solution into the flask by a 10 mL pipette;

9. Collecting cells in a 15 mL tube;

10. Washing the flask with CSM;

11. Aliquoting a given amount of the cells into CCB, using a yellow tip, followed by cell counting;

12. Inoculating the cells into a T75 flask after cell counting; and

13. Mixing the remaining cells and a cell stock solution in a Cryo tube and cryopreserving the mixture; and

CRM-P2 Medium Change Kit of the chondrocyte medium change and subculture process includes:

1. Taking a vinyl-wrapped CRM-P2 Medium Change Kit out of Cartilage RM Kit, spraying the Kit with a sufficient amount of a 70% alcohol, and transferring the Kit to a clean bench;

2. Removing a culture medium from a flask using a Pasteur pipette;

3. Aliquoting CPM into a 50 mL tube by a 25 mL pipette; and

4. Re-aliquoting a given amount of CPM from the aliquoted tube into the flask; and

CRM-P2 Subculture Kit of the chondrocyte medium change and subculture process includes:

1. Taking a vinyl-wrapped CRM-P2 Subculture Kit out of Cartilage RM Kit, spraying the Kit with a sufficient amount of a 70% alcohol, and transferring the Kit to a clean bench;

2. Pooling and mixing the culture medium of the T75 flask in a 50 mL tube;

3. Sampling a portion of the culture medium in a 50 mL tube and an E-tube;

4. Removing the remaining culture medium by a Pasteur pipette;

5. Aliquoting CSB-II into the flask, shaking to wash the flask, and removing a supernatant by a Pasteur pipette;

6. Repeating Step 5 three times;

7. Aliquoting a Pass solution into the flask by a 10 mL pipette, and maintaining the flask in an incubator for about 3 to 5 min;

8. Aliquoting a Neutro solution into the flask by a 10 mL pipette;

9. Collecting cells in a 15 mL tube;

10. Washing the flask with CSM under gentle shaking;

11. Aliquoting a given amount of the cells into CCB, using a yellow tip, followed by cell counting;

12. Inoculating the cells into a T150 flask after cell counting; and

13. Mixing the remaining cells and a cell stock solution in a Cryo tube and cryopreserving the mixture; and

CRM-P3 Medium Change Kit of the chondrocyte medium change and subculture process includes:

1. Taking a vinyl-wrapped CRM-P2 Medium Change Kit out of Cartilage RM Kit, spraying the Kit with a sufficient amount of a 70% alcohol, and transferring the Kit to a clean bench;

2. Removing the culture medium from the flask by a Pasteur pipette;

3. Aliquoting CPM into a 50 mL tube by a 25 mL pipette; and

4. Re-aliquoting a given amount of CPM from the aliquoted tube into the flask.

Further, CRM-Collection Kit of the chondrocyte therapy product preparation process which is applied to the present invention includes:

1. Taking a vinyl-wrapped CRM-Collection Kit out of Cartilage RM Kit, spraying the Kit with a sufficient amount of a 70% alcohol, and transferring the Kit to a clean bench;

2. Sampling a portion of the culture medium in a 50 mL tube using a 25 mL pipette, 3 days prior to delivery of a finished product;

3. On delivery day of the product, pooling and mixing the culture medium of a T150 flask in a 50 mL tube;

4. Sampling a portion of the culture medium in a 50 mL tube and an E-tube;

5. Removing the remaining culture medium in the flask, using a Pasteur pipette;

6. Aliquoting CSB-II into the flask by a 25 mL pipette, followed by washing;

7. Repeating Step 6 three times;

8. Aliquoting a Pass solution into the flask by a 10 mL pipette, and maintaining the flask in an incubator for about 4 to 5 min;

9. Aliquoting a Neutro solution into the flask by a 10 mL pipette;

10. Placing cell strainers over two 50 mL tubes, respectively;

11. Aliquoting the Pass solution-treated suspension in the flask into the tubes over which the cell strainers were placed, using a 25 mL pipette;

12. Aliquoting the cells into CCB using a yellow tip, and performing cell count a total of three times;

13. Aliquoting CCM into a 1 mL vial, and mixing the cells and CCM, using a filter blue tip; and

14. Assembling a rubber cap and an aluminum cap into a 1 mL vial; and

CRM-Delivery Kit & CRM-Medium Packaging Kit of the chondrocyte therapy product preparation process includes:

1. Unwrapping an outer paper packaging of CRM-Delivery Kit and CRM-Medium Packaging Kit contained in Cartilage RM Kit;

2. Transferring a styrofoam box with an inner packaging only to a surgical room, while leaving the removed outer packaging; and

3. Taking vials No. 1 and No. 4 out of the box transferred into the surgical room, and mixing the vials and CRM-Implantation Kit for being ready to use; and

CRM-Implantation Kit the chondrocyte therapy product preparation process includes:

1. Using a mixing tip inside CRM-Implantation Kit, mixing a medium and a substrate for use in admixture with the chondrocyte therapy product in a device 2 set; and

2. Confirming full dissolution of the mixed substrate and medium composition, attaching an 18-gauge needle to the syringe, and injecting the composition into the cartilage defect area of the patient.

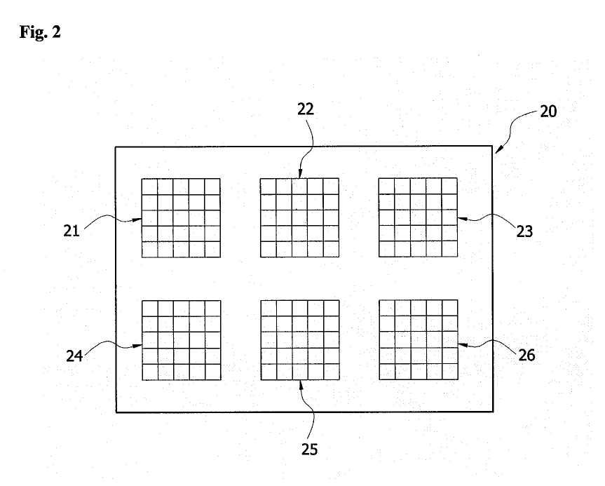

On the other hand, the present invention can also be constituted as shown in FIG. 2.

That is, as shown in FIG. 2, an aseptic/sterile kit for bone regeneration is provided according to the present invention. The kit includes a bone regeneration kit 20 in a configuration that each process can be performed according to the functionally-specialized kit sets for each step, via division of the overall processes into the corresponding steps for isolation, culture, collection and storage of cells, and transplantation of the desired cells into the target sites of the body. The bone regeneration kit 20 includes a bone marrow collection kit 21, an osteoblast isolation kit 22, an osteoblast medium change and subculture kit 23, and an osteoblast therapy product preparation kit 24.

Herein, the bone marrow collection kit 21 includes a Universal container which is an internal container for tissue delivery, a blue tip used to take 1 mL of a solution, a styrofoam box which is an internal box for tissue delivery under refrigeration conditions, sponge used for introduction of a transport bottle into the styrofoam box, and a Koolit refrigerant to keep cold conditions.

In addition, the osteoblast isolation kit 22 includes a Pasteur pipette for solution removal, a cell strainer which is placed over a centrifuge tube and separates cells from the solution by passing the resulting suspension of cells and culture medium through it, a tube for centrifugation to wash cells, an E-tube used upon mixing trypan blue and the cell-containing medium for cell counting, a blue tip to take 1 mL of a solution, a pipette for solution transfer, a T75 cell culture flask, and a cryo tube for cryopreservation of cells.

In addition, the osteoblast medium change and subculture kit 23, which is applied to the present invention, includes a Pasteur pipette for solution removal, a tube for centrifugation to wash cells, a pipette for solution transfer, a pipette for transfer of a large volume of solution, a Yellow tip for taking a small volume of a solution, an E-tube used upon mixing trypan blue and the cell-containing medium, a T75 cell culture flask, and a cryo tube for cryopreservation of cells.

The osteoblast therapy product preparation kit 24 according to the present invention includes a Pasteur pipette for solution removal, a cryo tube for cryopreservation of cells, an E-tube used upon mixing trypan blue and the cell-containing medium for cell counting, a tube for centrifugation to wash cells, a pipette for solution transfer, a cell strainer which is placed over a centrifuge tube and separates cells from the solution by passing the resulting suspension of cells and the culture medium through it, a filter blue tip to take a solution, a rubber cap and an aluminum cap for stopping a vial, a 1 mL V-vial used to contain cells for preparation of an osteoblast therapy product, a styrofoam box which is an internal box for delivery of the osteoblast therapy product under refrigeration conditions, sponge used for introduction of the osteoblast therapy product-containing vial into the styrofoam box, and a Koolit refrigerant to keep cold conditions.

Further, the bone regeneration kit 20 also includes a media kit 25 for isolation/culture/preparation/cryopreservation of osteoblasts and a media kit 26 for isolation/culture/cryopreservation of osteoblasts.

The present invention, as constituted above, achieves regeneration of the bone by bone marrow collection; osteoblast isolation; osteoblast medium change and subculture; and osteoblast therapy product preparation, using the bone regeneration kit 20. The example of the present invention will be described hereinafter.

### Example 2

The bone marrow collection process includes:

1. Unwrapping an outer paper packaging of BRM-Transport Kit contained in Bone RM Kit;

2. Transferring a styrofoam box with an inner packaging only to a surgical room, while leaving the removed outer packaging; and

3. Placing the bone marrow in a bone marrow tissue transport container containing a red medium inside the styrofoam box, double-wrapping of the bone marrow tissue transport container with a styrofoam box and an outer paper box and then transferring the package into a cell culture room.

In addition, BRM-Initiation Kit of the osteoblast isolation process includes:

1. Taking BRM-Isolation Kit out of the BRM-Initiation Kit, spraying the Kit with a sufficient amount of a 70% alcohol, and transferring the Kit to a clean bench;

2. Weighing the bone marrow tissues transported on the BRM-Transport Kit, in the clean bench, using a 50 mL tube;

3. Transferring the bone marrow tissues into a 50 mL tube, aliquoting BSB-I into the tube using a 25 mL pipette, and washing the bone marrow;

4. Removing the bone marrow tissue-washed solution by a Pasteur pipette and repeating Step 3;

5. Adding a BDB solution to the washed bone marrow tissues, using a 25 mL pipette;

6. After a predetermined period of time, neutralizing the bone marrow tissues with a given amount of the BDB solution by using a 10 mL pipette;

7. After neutralization, re-washing the neutralized bone marrow tissues with a BSM solution; and

8. Transferring the washed-nucleated cells into a T75 flask and maintaining the cells in a CO₂ incubator at 37° C.; and

BRM-Primary Kit of the osteoblast isolation process includes:

1. Taking a vinyl-wrapped BRM-Primary Kit out of Bone RM Kit, spraying the Kit with a sufficient amount of a 70% alcohol, and transferring the Kit to a clean bench;

2. Transferring the suspended cell layer of the T75 flask, placed in the CO₂ incubator at 37° C., into a 50 mL tube by a 10 mL pipette; and

3. Centrifuging the 50 mL tube, mixing the cells and a BSM solution using a 25 mL pipette, and inoculating the mixture into the T75 flask.

In addition, BRM-P1 Medium Change Kit of the osteoblast medium change and subculture process of the present invention includes:

1. Taking a vinyl-wrapped BRM-P1 Medium Change Kit out of Bone RM Kit, spraying the Kit with a sufficient amount of a 70% alcohol, and transferring the Kit to a clean bench;

2. Removing a culture medium inside a flask using a Pasteur pipette;

3. Aliquoting BSM into a 50 mL tube by a 25 mL pipette; and

4. Re-aliquoting a given amount of BSM from the aliquoted tube into the flask; and

BRM-P1 Subculture Kit of the osteoblast medium change and subculture process includes:

1. Taking a vinyl-wrapped BRM-P1 Subculture Kit out of Bone RM Kit, spraying the Kit with a sufficient amount of a 70% alcohol, and transferring the Kit to a clean bench;

2. Pooling and mixing the culture medium of the T25 flask in a 50 mL tube;

3. Sampling a portion of the culture medium in a 50 mL tube and an E-tube;

4. Removing the remaining culture medium by a Pasteur pipette;

5. Aliquoting BSM-II into the flask, shaking to wash the flask, and removing a supernatant by a Pasteur pipette;

6. Repeating Step 5 three times;

7. Aliquoting a Pass solution into the flask by a 10 mL pipette, and maintaining the flask in an incubator for about 3 min;

8. Aliquoting a Neutro solution into the flask by a 10 mL pipette;

9. Collecting cells in a 15 mL tube;

10. Washing the flask with BSM;

11. Aliquoting a given amount of the cells into BCB, using a yellow tip, followed by viable cell counting;

12. Inoculating the cells into a T75 flask after viable cell counting; and

13. Mixing the remaining cells and a cell stock solution in a Cryo tube and cryopreserving the mixture; and

BRM-P2 Medium Change Kit of the osteoblast medium change and subculture process includes:

1. Taking a vinyl-wrapped BRM-P2 Medium Change Kit out of Bone RM Kit, spraying the Kit with a sufficient amount of a 70% alcohol, and transferring the Kit to a clean bench;

2. Removing a culture medium from a flask using a Pasteur pipette;

3. Aliquoting BPM into a 50 mL tube by a 25 mL pipette; and

4. Re-aliquoting a given amount of BPM from the aliquoted tube into the flask; and

BRM-P2 Subculture Kit of the osteoblast medium change and subculture process includes:

1. Taking a vinyl-wrapped BRM-P2 Subculture Kit out of Bone RM Kit, spraying the Kit with a sufficient amount of a 70% alcohol, and transferring the Kit to a clean bench;

2. Pooling and mixing the culture medium of a T75 flask in a 50 mL tube;

3. Sampling a portion of the culture medium in a 50 mL tube and an E-tube;

4. Removing the remaining culture medium by a Pasteur pipette;

5. Aliquoting BSB-II into the flask, shaking to wash the flask, and removing a supernatant by a Pasteur pipette;

6. Repeating Step 5 three times;

7. Aliquoting a Pass solution into the flask by a 10 mL pipette, and maintaining the flask in an incubator for about 3 to 5 min;

8. Aliquoting a Neutro solution into the flask by a 10 mL pipette;

9. Collecting cells in a 15 mL tube;

10. Washing the flask with BSM under gentle shaking;

11. Aliquoting a given amount of the cells into BCB, using a yellow tip, followed by cell counting;

12. Inoculating the cells into a T150 flask after cell counting; and

13. Mixing the remaining cells and a cell stock solution in a Cryo tube and cryopreserving the mixture; and

BRM-P3 Medium Change Kit of the osteoblast medium change and subculture process includes:

1. Taking a vinyl-wrapped BRM-P2 Medium Change Kit out of Bone RM Kit, spraying the Kit with a sufficient amount of a 70% alcohol, and transferring the Kit to a clean bench;

2. Removing the culture medium from the flask using a Pasteur pipette;

3. Aliquoting BPM into a 50 mL tube by a 25 mL pipette; and

4. Re-aliquoting a given amount of BPM from the aliquoted tube into the flask.

Further, BRM-Collection Kit of the osteoblast therapy product preparation process according to the present invention includes:

1. Taking a vinyl-wrapped BRM-Collection Kit out of Bone RM Kit, spraying the Kit with a sufficient amount of a 70% alcohol, and transferring the Kit to a clean bench;

2. Sampling a portion of the culture medium in a 50 mL tube using a 25 mL pipette, 3 days prior to delivery of a finished product;

3. On delivery day of the product, pooling and mixing the culture medium of the T150 flask in a 50 mL tube;

4. Sampling a portion of the culture medium in a 50 mL tube and an E-tube;

5. Removing the remaining culture medium in the flask, using a Pasteur pipette;

6. Aliquoting BSB-II into the flask by a 25 mL pipette, followed by washing;

7. Repeating Step 6 three times;

8. Aliquoting a Pass solution into the flask by a 10 mL pipette, and maintaining the flask in an incubator for about 4 to 5 min;

9. Aliquoting a Neutro solution into the flask by a 10 mL pipette;

10. Placing cell strainers over two 50 mL tubes, respectively;

11. Aliquoting the Pass solution-treated suspensions in the flasks into the tubes over which the cell strainers were placed, using a 25 mL pipette;

12. Aliquoting the cells into BCB using a yellow tip, and performing cell count a total of three times;

13. Aliquoting the BCM into a 1 mL vial, and mixing the cells and BCM, using a filter blue tip; and

14. Assembling a rubber cap and an aluminum cap into the 1 mL vial; and

BRM-Delivery Kit & BRM-Medium Packaging Kit of the osteoblast therapy product preparation process includes:

1. Unwrapping an outer paper packaging of BRM-Delivery Kit and BRM-Medium Packaging Kit contained in Bone RM Kit;

2. Transferring a styrofoam box with an inner packaging only to a surgical room, while leaving the removed outer packaging; and

3. Taking vials No. 1 and No. 4 out of the box transferred into the surgical room, and mixing the vials and BRM-Implantation Kit for being ready to use; and

BRM-Implantation Kit of the osteoblast therapy product preparation process:

1. Using a mixing tip inside BRM-Implantation Kit, mixing a medium and a substrate for use in admixture with the osteoblast therapy product in a device 2 set; and

2. Confirming full dissolution of the mixed substrate and medium composition, attaching a needle to the syringe, and injecting the composition into the bone defect site of the patient.

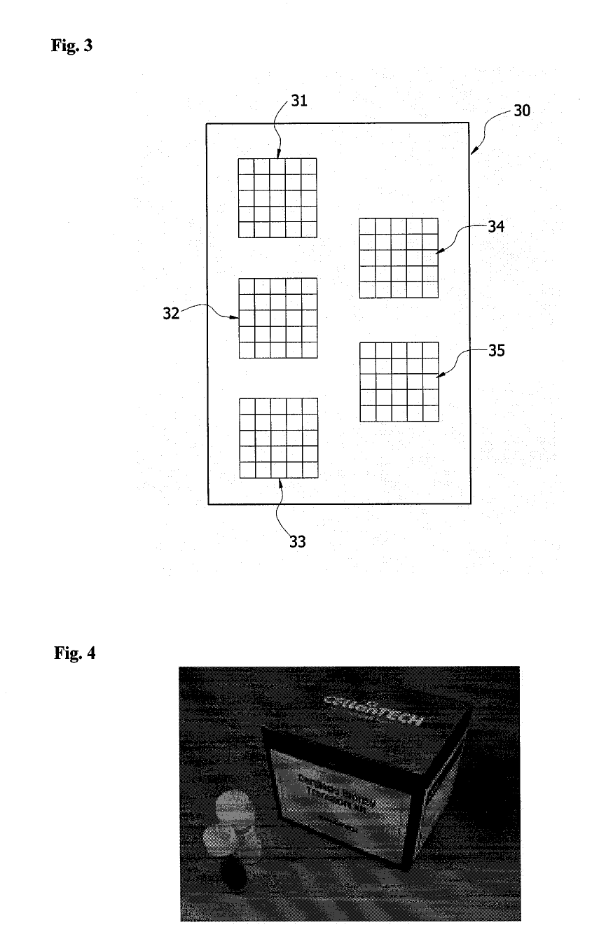

Alternatively, the present invention can also be constituted as shown in FIG. 3.

That is, as shown in FIG. 3, an aseptic/sterile kit for storage of umbilical cord blood is provided according to the present invention. The kit includes an umbilical cord blood storage kit 30 in a configuration that each process can be performed according to the functionally-specialized kit sets for each step, via division of the overall processes into the corresponding steps for isolation, culture, collection and storage of cells, and transplantation of the desired cells into the target sites of the body. The umbilical cord blood storage kit includes an umbilical cord blood collection kit 31, a hematopoietic stem cell isolation kit 32, and a hematopoietic stem cell cryopreservation kit 33.

Herein, the umbilical cord blood collection kit 31 is provided with a blood collection bag for collection of large amounts of blood, a file case for delivery of the blood collection bag, a zipper bag for storage of the blood collection bag until transport of the bag, and a sticker attached to a surface of the file case.

In addition, the hematopoietic stem cell isolation kit 32 is provided with a processing bag for concentration of hematopoietic stem cell after removal of red blood cells, a syringe used upon sample extraction intended for quality control, a cell strainer which is placed over a centrifuge tube and separates cells from the solution by passing the resulting suspension of cells and a culture medium through it, a tube for centrifugation to wash cells, latex gloves for conducting aseptic processing operations, a CCZ vial to contain cells for storage of hematopoietic stem cells, an aluminum seal for stopping the vial, a blue tip used to take a solution, and an E-tube used upon mixing of trypan blue and the cell-containing medium.

In addition, the hematopoietic stem cell cryopreservation kit 33, which is applied to the present invention, includes a freezing bag which is a tool used to freeze the hematopoietic stem cells, a cryowrap for protecting the freezing bag, and a canister for keeping the freezing bag and the cryowrap.

The umbilical cord blood storage kit 30, which is applied to the present invention, includes a medium kit 34 for use in isolation and cryopreservation of the hematopoietic stem cells and a medium kit 35 for use in isolation and cryopreservation of the hematopoietic stem cells.

The present invention, as constituted above, achieves storage of the umbilical cord blood by umbilical cord blood collection; hematopoietic stem cell isolation; and hematopoietic stem cell cryopreservation, using the umbilical cord blood storage kit. An example of the present invention will be described hereinafter.

### Example 3

The umbilical cord blood collection process includes:

1. Transporting a file case of USC RM-Transport Kit contained in USC RM Kit to a surgical room;

2. Opening a zipper bag of the transported file case, and collecting umbilical cord blood using a sterile blood collection bag; and

3. Placing the collected blood in the file case of USC RM-Transport Kit and transferring the blood to a processing room.

In addition, the hematopoietic stem cell isolation process includes:

1. Withdrawing a given amount of blood from the blood collection bag, using a 10 mL syringe, and aliquoting the blood into an EPP tube;

2. Adding a given amount of a UES solution to the tube, using a 30 mL syringe, followed by stirring and allowing to stand at room temperature;

3. After allowing to stand for a certain period of time, separating a primary plasma layer into the processing bag in a semi-automatic plasma separator;

4. Repeating Steps 2 and 3;

5. Attaching the processing bag to a bucket, followed by weighing and centrifugation;

6. Detaching the processing bag from the bucket after centrifugation;

7. Mounting the processing bag to an automatic plasma separator;

8. Aliquoting the plasma into a 15 mL tube, using the automatic plasma separator;

9. Placing the remaining plasma into a 50 mL tube, using the automatic plasma separator; and

10. Withdrawing the plasma from the 50 mL tube using a 10 mL syringe, and aliquoting the plasma into a vacumtainer.

Further, the hematopoietic stem cell cryopreservation process includes:

1. Suspending a processing bag, withdrawing the contents from the suspended processing bag by a 10 mL syringe and aliquoting the contents into an EPP;

2. Adding a cell stock solution to the processing bag suspended in ice-cold water by a 10 mL syringe, followed by intimate mixing;

3. Sterilizing an outer surface of the processing bag with a 70% alcohol;

4. Connecting a freezing bag to the processing bag, thereby receiving a concentrated layer of nucleated cells and removing air bubbles;

5. Placing the freezing bag in a double-wrapped bag, followed by compression packaging and sterilization; and

6. Inserting the freezing bag packaged in a double-wrapped bag into the canister.

### INDUSTRIAL APPLICABILITY

As apparent from the above description, the present invention provides a method of using a kit associated with the preparation and implantation of a cell therapy product intended for regeneration of cartilage and bone and storage of umbilical cord blood, and the treatment of the umbilical cord blood. In particular, the present invention enables construction and supply of various materials, culture media and solutions, necessary for isolation, culture, collection, implantation and storage processes of desired cells, in the form of a single kit for easy and convenient use. Hence, the present invention can realize reduction of problems such as high production costs, a long period of production time and high labor costs in the preparation of cell therapy products and the storage of umbilical cord blood, suffered by conventional arts.

In addition, the kit of the present invention is an aseptic and sterile medical kit which enables safe isolation, culture, collection and storage of cells from the collected cartilage, bone marrow tissues and umbilical cord blood, and implantation of the desired cells into the target sites of the body. For this purpose, the medical kit is configured in the form of a single kit set, such that each process can be performed according to the functionally-specialized kit sets for corresponding steps, via division of the overall process into the corresponding steps.

Further, the present invention realizes standardization of all processes, associated with the preparation of a cell therapy product intended for regeneration of cartilage and bone, according to the corresponding steps, and fabrication of a kit suited for the standardized processes. Therefore, the present invention enables easy and convenient use of the kit at a time, which leads to activation and promotion of production of the cell therapy products and further, commercialization of a novel therapy using the tissue engineering.

In addition, the present invention also enables standardization of a protocol for storage of umbilical cord blood, which has the same configuration as illustrated in the above-mentioned kit for cartilage and bone regeneration, fabrication of functionally-specialized kits corresponding to each process, and consequently convenient use thereof at a time. Hence, as compared to conventional storage/treatment methods, reduction of costs, time and manpower associated with the storage of the umbilical cord blood can be achieved.

As a result, the present invention accomplishes remarkably improved quality and reliability of the products and thereby is very useful to enhance customer satisfaction.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1-31. (canceled)

32. A cartilage regeneration of an aseptic/sterile medical kit comprising a cartilage regeneration kit (10) in a configuration that each process can be performed according to the functionally-specialized kit sets for each step, via division of the overall processes into the corresponding steps for isolation, culture, collection and storage of cells, and implantation of the desired cells into the target sites of the body, wherein the cartilage regeneration kit (10) includes a cartilage tissue collection kit (11), a chondrocyte isolation kit (12), a chondrocyte medium change and subculture kit (13), and a chondrocyte therapy product preparation kit (14),
wherein the cartilage tissue collection kit (11) includes a hard tissue collection kit for collection of cartilage tissues, a universal container which is an internal container for tissue delivery, a transport bottle which is an external container for tissue delivery, a blue tip (1 mL) used to take 1 mL of a solution, a styrofoam box which is an internal box for tissue delivery under refrigeration conditions, sponge used for introduction of the transport bottle into the styrofoam box, and a Koolit refrigerant to keep the cold conditions,
wherein the chondrocyte isolation kit (12) includes a Pasteur pipette for solution removal, T25 (plug seal) as a cell culture vessel with a plug-seal cap (a 25 cm² flask), a cell strainer which is placed over a 50 mL centrifuge tube and separates cells from the solution by passing the resulting suspension of cells and culture medium through it, a tube for centrifugation to wash cells, an E-tube used upon mixing trypan blue and the cell-containing medium, a blue tip to take 1 mL of a solution, a pipette for solution transfer, a syringe filter for solution filtration, a T25 cell culture flask, and a cryo tube for cryopreservation of cells,
wherein the chondrocyte medium change and subculture kit (13), includes a Pasteur pipette for solution removal, a tube for centrifugation to wash cells, a pipette for solution transfer, a pipette for transfer of a large volume of a solution, a Yellow tip for taking a small volume of a solution, an E-tube used upon mixing trypan blue and the cell-containing medium, a T75 cell culture flask, and a cryo tube for cryopreservation of cells, and
wherein the chondrocyte therapy product preparation kit (14) includes a Pasteur pipette for solution removal, a cryo tube for cryopreservation of cells, an E-tube used upon mixing trypan blue and the cell-containing medium, a tube for centrifugation to wash cells, a pipette for solution transfer, a pipette for transfer of a large volume of solution, a cell strainer which is placed over a 50 mL centrifuge tube and separates cells from the solution by passing the resulting suspension of cells and culture medium through it, a filter blue tip to take a solution, a rubber cap and an aluminum cap for stopping a vial, a 1 mL V-vial used to contain cells for preparation of a chondrocyte therapy product, a styrofoam box which is an internal box for tissue delivery under refrigeration conditions, sponge used for introduction of the chondrocyte therapy product-containing vial into the styrofoam box, a Koolit refrigerant to keep cold conditions and device 2 sets for implantation of cells into a defect area.

33. The cartilage regeneration medical kit according to claim 32, wherein the cartilage regeneration kit (10) further includes a media kit (15) for isolation/culture/preparation/cryopreservation of cells and a media kit (16) for isolation/culture/cryopreservation of cells.

34. The cartilage regeneration medical kit according to claim 32, a method of using a cartilage regeneration kit, for regeneration of cartilage by cartilage tissue collection; chondrocyte isolation; chondrocyte medium change and subculture; preparation of a chondrocyte therapy product; media for isolation/culture/preparation/cryopreservation of cells; and media for isolation/culture/cryopreservation of cells

35. The method according to claim 34, wherein the cartilage tissue collection process includes:
unwrapping an outer paper packaging of CRM-Transport Kit contained in Cartilage RM Kit;
transferring a styrofoam box with an inner packaging only to a surgical room, while leaving the removed outer packaging; and
collecting cartilage tissues using a sterile hard tissue collector inside the styrofoam box, placing the collected cartilage tissues in a cartilage tissue transport container containing a red medium, double-wrapping of the cartilage tissue transport container with a styrofoam box and an outer paper box, and then transferring the package into a cell culture room.

36. The method according to claim 34, wherein CRM-Initiation Kit of the chondrocyte isolation process includes:
(1) taking CRM-Isolation Kit out of the CRM-Initiation Kit, spraying the Kit with a sufficient amount of a 70% alcohol, and transferring the Kit to a clean bench;
(2) weighing the cartilage tissues transported on the CRM-Transport Kit, in the clean bench, using a 50 mL tube;
(3) transferring the cartilage tissues into a 50 mL tube, aliquoting CSB-I into the tube, using a 25 mL pipette, and washing the cartilage tissues;
(4) removing the cartilage tissue-washed solution by a Pasteur pipette and repeating previous step 3;
(5) transferring the washed cartilage tissues into a dish and slicing the tissues into pieces using blades No. 10 and 11;
(6) transferring the sliced cartilage tissues into a 15 mL tube;
(7) aliquoting CSB-I into the tube using a 10 mL pipette;
(8) centrifuging the cartilage tissue-containing tube at 1200 rpm for 1 min;
(9) washing the tissues and removing a supernatant by a Pasteur pipette;
(10) repeating the previous steps (8 and 9) three times;
(11) providing two T25 flasks (plug seal), and using a 10 mL pipette, aliquoting Cartisepor into one T25 flask and aliquoting a mixture of Cartisepor and an antibiotic into the other flask;
(12) transferring ⅓ of the washed cartilage tissue into the flask containing CSB-I, and transferring the remaining ⅔ of the washed cartilage tissue into the flask containing CSB-II;
(13) after transferring the tissues into the flasks, incubating them in a CO2 incubator at 37° C., for 12 to 16 hours and stirring them at 37° C. and 100 rpm for 30 min; and
(14) placing cell strainers over 50 mL tubes, and filtering suspensions in the flasks, and
CRM-Primary Kit of the chondrocyte isolation process includes,
(1) taking a vinyl-wrapped CRM-Primary Kit out of Cartilage RM Kit, spraying the Kit with a sufficient amount of a 70% alcohol, and transferring the Kit to a clean bench;
(2) aliquoting CSM into a 50 mL tube by a 10 mL pipette;
(3) centrifuging and washing the contents of the tube, removing a supernatant by a Pasteur pipette, aliquoting CSM again into the tube, followed by washing;
(4) mixing 1/20 of cells and PCF in a cryo tube and cryopreserving the mixture; and
(5) on the following day, recovering suspension cells non-adhered to the T25 flask in a 15 mL tube and inoculating the cells into the T25 flask.

37. The method according to claim 34, wherein CRM-P1 Medium Change Kit of the chondrocyte medium change and subculture process includes:
(1) taking a vinyl-wrapped CRM-P1 Medium Change Kit out of Cartilage RM Kit, spraying the Kit with a sufficient amount of a 70% alcohol, and transferring the Kit to a clean bench;
(2) removing a culture medium inside a flask using a Pasteur pipette;
(3) aliquoting CSM into a 50 mL tube by a 25 mL pipette; and
(4) re-aliquoting a given amount of CSM from the aliquoted tube into the flask; and
CRM-P1 Subculture Kit of the chondrocyte medium change and subculture process includes:
(1) taking a vinyl-wrapped CRM-P1 Subculture Kit out of Cartilage RM Kit, spraying the Kit with a sufficient amount of a 70% alcohol, and transferring the Kit to a clean bench;
(2) pooling and mixing the culture medium of T25 flask in a 50 mL tube;
(3) sampling a portion of the culture medium in a 50 mL tube and an E-tube;
(4) removing the remaining culture medium by a Pasteur pipette;
(5) aliquoting CSB-II into the flask, shaking to wash the flask, and removing a supernatant by a Pasteur pipette;
(6) repeating the previous step (5) three times;
(7) aliquoting a Pass solution into the flask by a 10 mL pipette, and maintaining the flask in an incubator for about 3 min;
(8) aliquoting a Neutro solution into the flask by a 10 mL pipette;
(9) collecting cells in a 15 mL tube;
(10) washing the flask with CSM;
(11) aliquoting a given amount of the cells into CCB, using a yellow tip, followed by cell counting;
(12) inoculating the cells into a T75 flask after cell counting; and
(13) mixing the remaining cells and a cell stock solution in a Cryo tube and cryopreserving the mixture; and
CRM-P2 Medium Change Kit of the chondrocyte medium change and subculture process includes:
(1) taking a vinyl-wrapped CRM-P2 Medium Change Kit out of Cartilage RM Kit, spraying the Kit with a sufficient amount of a 70% alcohol, and transferring the Kit to a clean bench;
(2) removing a culture medium from a flask using a Pasteur pipette;
(3) aliquoting CPM into a 50 mL tube by a 25 mL pipette; and
(4) re-aliquoting a given amount of CPM from the aliquoted tube into the flask; and
CRM-P2 Subculture Kit of the chondrocyte medium change and subculture process includes:
(1) taking a vinyl-wrapped CRM-P2 Subculture Kit out of Cartilage RM Kit, spraying the Kit with a sufficient amount of a 70% alcohol, and transferring the Kit to a clean bench;
(2) pooling and mixing the culture medium of the T75 flask in a 50 mL tube;
(3) sampling a portion of the culture medium in a 50 mL tube and an E-tube;
(4) removing the remaining culture medium by a Pasteur pipette;
(5) aliquoting CSB-II into the flask, shaking to wash the flask, and removing a supernatant by a Pasteur pipette;
(6) repeating the previous step (5) three times;
(7) aliquoting a Pass solution into the flask by a 10 mL pipette, and maintaining the flask in an incubator for about 3 to 5 min;
(8) aliquoting a Neutro solution into the flask by a 10 mL pipette;
(9) collecting cells in a 15 mL tube;
(10) washing the flask with CSM under gentle shaking;
(11) aliquoting a given amount of the cells into CCB, using a yellow tip, followed by cell counting;
(12) inoculating the cells into a T150 flask after cell counting; and
(13) mixing the remaining cells and a cell stock solution in a Cryo tube and cryopreserving the mixture; and
CRM-P3 Medium Change Kit of the chondrocyte medium change and subculture process includes:
(1) taking a vinyl-wrapped CRM-P2 Medium Change Kit out of Cartilage RM Kit, spraying the Kit with a sufficient amount of a 70% alcohol, and transferring the Kit to a clean bench;
(2) removing the culture medium from the flask by a Pasteur pipette;
(3) aliquoting CPM into a 50 mL tube by a 25 mL pipette; and
(4) re-aliquoting a given amount of CPM from the aliquoted tube into the flask.

38. The method according to claim 34, wherein CRM-Collection Kit of the chondrocyte therapy product preparation process includes:
(1) taking a vinyl-wrapped CRM-Collection Kit out of Cartilage RM Kit, spraying the Kit with a sufficient amount of a 70% alcohol, and transferring the Kit to a clean bench;
(2) sampling a portion of the culture medium in a 50 mL tube using a 25 mL pipette, 3 days prior to delivery of a finished product;
(3) on delivery day of the product, pooling and mixing the culture medium of a T150 flask in a 50 mL tube;
(4) sampling a portion of the culture medium in a 50 mL tube and an E-tube;
(5) removing the remaining culture medium in the flask, using a Pasteur pipette;
(6) aliquoting CSB-II into the flask by a 25 mL pipette, followed by washing;
(7) repeating the previous step (6) three times;
(8) aliquoting a Pass solution into the flask by a 10 mL pipette, and maintaining the flask in an incubator for about 4 to 5 min;
(9) aliquoting a Neutro solution into the flask by a 10 mL pipette;
(10) placing cell strainers over two 50 mL tubes, respectively;
(11) aliquoting the Pass solution-treated suspension in the flask into the tubes over which the cell strainers were placed, using a 25 mL pipette;
(12) aliquoting the cells into CCB using a yellow tip, and performing cell count a total of three times;
(13) aliquoting CCM into a 1 mL vial, and mixing the cells and CCM, using a filter blue tip; and
(14) assembling a rubber cap and an aluminum cap into a 1 mL vial; and
CRM-Delivery Kit & CRM-Medium Packaging Kit of the chondrocyte therapy product preparation process includes:
(1) unwrapping an outer paper packaging of CRM-Delivery Kit and CRM-Medium Packaging Kit contained in Cartilage RM Kit;
(2) transferring a styrofoam box with an inner packaging only to a surgical room, while leaving the removed outer packaging; and
(3) taking vials No. 1 and No. 4 out of the box transferred into the surgical room, and mixing the vials and CRM-Implantation Kit for being ready to use; and
CRM-Implantation Kit of the chondrocyte therapy product preparation process includes:
(1) using a mixing tip inside CRM-Implantation Kit, mixing a medium and a substrate for use in admixture with the chondrocyte therapy product in a device 2 set; and
(2) confirming full dissolution of the mixed substrate and medium composition, attaching an 18-gauge needle to the syringe, and injecting the composition into the cartilage defect area of the patient.

39. An aseptic/sterile kit of bone regeneration comprising a bone regeneration kit (20) in a configuration that each process can be performed according to the functionally-specialized kit sets for each step, via division of the overall processes into the corresponding steps for isolation, culture, collection and storage of cells, and transplantation of the desired cells into the target sites of the body, wherein the bone regeneration kit (20) includes a bone marrow collection kit (21), an osteoblast isolation kit (22), an osteoblast medium change and subculture kit (23), and an osteoblast therapy product preparation kit (24),
wherein the bone marrow collection kit (21) includes a universal container which is an internal container for tissue delivery, a blue tip used to take 1 mL of a solution, a styrofoam box which is an internal box for tissue delivery under refrigeration conditions, sponge used for introduction of a transport bottle into the styrofoam box, and a Koolit refrigerant to keep cold conditions,
wherein the osteoblast isolation kit (22) includes a Pasteur pipette for solution removal, a cell strainer which is placed over a centrifuge tube and separates cells from the solution by passing the resulting suspension of cells and culture medium through it, a tube for centrifugation to wash cells, an E-tube used upon mixing trypan blue and the cell-containing medium for cell counting, a blue tip to take 1 mL of a solution, a pipette for solution transfer, a T75 cell culture flask, and a cryo tube for cryopreservation of cells,
wherein the osteoblast medium change and subculture kit (23) includes a Pasteur pipette for solution removal, a tube for centrifugation to wash cells, a pipette for solution transfer, a pipette for transfer of a large volume of solution, a Yellow tip for taking a small volume of a solution, an E-tube used upon mixing trypan blue and the cell-containing medium, a T75 cell culture flask, and a cryo tube for cryopreservation of cells, and
wherein the osteoblast therapy product preparation kit (24) includes a Pasteur pipette for solution removal, a cryo tube for cryopreservation of cells, an E-tube used upon mixing trypan blue and the cell-containing medium for cell counting, a tube for centrifugation to wash cells, a pipette for solution transfer, a cell strainer which is placed over a centrifuge tube and separates cells from the solution by passing the resulting suspension of cells and the culture medium through it, a filter blue tip to take a solution, a rubber cap and an aluminum cap for stopping a vial, a 1 mL V-vial used to contain cells for preparation of an osteoblast therapy product, a styrofoam box which is an internal box for delivery of the osteoblast therapy product under refrigeration conditions, sponge used for introduction of the osteoblast therapy product-containing vial into the styrofoam box, and a Koolit refrigerant to keep cold conditions.

40. The bone regeneration aseptic/sterile kit according to claim 39, wherein the bone regeneration kit (20) also includes a media kit (25) for isolation/culture/preparation/cryopreservation of osteoblasts and a media kit (26) for isolation/culture/cryopreservation of osteoblasts.

41. The bone regeneration aseptic/sterile kit according to claim 39, a method of using bone regeneration kit (20), for regeneration of the bone by bone marrow collection; osteoblast isolation; osteoblast medium change and subculture; and osteoblast therapy product preparation.

42. The method according to claim 41, wherein the bone marrow collection process includes:
(1) unwrapping an outer paper packaging of BRM-Transport Kit contained in Bone RM Kit;
(2) transferring a styrofoam box with an inner packaging only to a surgical room, while leaving the removed outer packaging; and
(3) placing the bone marrow in a bone marrow tissue transport container containing a red medium inside the styrofoam box, double-wrapping of the bone marrow tissue transport container with a styrofoam box and an outer paper box and then transferring the package into a cell culture room.

43. The method according to claim 41, wherein BRM-Initiation Kit of the osteoblast isolation process includes:
(1) taking BRM-Isolation Kit out of the BRM-Initiation Kit, spraying the Kit with a sufficient amount of a 70% alcohol, and transferring the Kit to a clean bench;
(2) weighing the bone marrow tissues transported on the BRM-Transport Kit, in the clean bench, using a 50 mL tube;
(3) transferring the bone marrow tissues into a 50 mL tube, aliquoting BSB-I into the tube using a 25 mL pipette, and washing the bone marrow;
(4) removing the bone marrow tissue-washed solution by a Pasteur pipette and repeating previous step (3);
(5) adding a BDB solution to the washed bone marrow tissues, using a 25 mL pipette;
(6) after a predetermined period of time, withdrawing a given amount of the BDB solution by using a 10 mL pipette and neutralizing the bone marrow tissues with the BDB solution;
(7) after neutralization, re-washing the neutralized bone marrow tissues with a BSM solution; and
(8) transferring the washed-nucleated cells into a T75 flask and maintaining the cells in the CO2 incubator at 37° C.; and
BRM-Primary Kit of the osteoblast isolation process includes:
(1) taking a vinyl-wrapped BRM-Primary Kit out of Bone RM Kit, spraying the Kit with a sufficient amount of a 70% alcohol, and transferring the Kit to a clean bench;
(2) transferring the suspended cell layer of the T75 flask, placed in a CO2 incubator at 37° C., into a 50 mL tube by a 10 mL pipette; and
(3) centrifuging the 50 mL tube, mixing the cells and a BSM solution using a 25 mL pipette, and inoculating the mixture into the T75 flask.

44. The method according to claim 41, wherein BRM-P1 Medium Change Kit of the osteoblast medium change and subculture process includes:
(1) taking a vinyl-wrapped BRM-P1 Medium Change Kit out of Bone RM Kit, spraying the Kit with a sufficient amount of a 70% alcohol, and transferring the Kit to a clean bench;
(2) removing a culture medium inside a flask using a Pasteur pipette;
(3) aliquoting BSM into a 50 mL tube by a 25 mL pipette; and
(4) re-aliquoting a given amount of BSM from the aliquoted tube into the flask; and
BRM-P1 Subculture Kit of the osteoblast medium change and subculture process includes:
(1) taking a vinyl-wrapped BRM-P1 Subculture Kit out of Bone RM Kit, spraying the Kit with a sufficient amount of a 70% alcohol, and transferring the Kit to a clean bench;
(2) pooling and mixing the culture medium of the T25 flask in a 50 mL tube;
(3) sampling a portion of the culture medium in a 50 mL tube and an E-tube;
(4) removing the remaining culture medium by a Pasteur pipette;
(5) aliquoting BSM-II into the flask, shaking to wash the flask, and removing a supernatant by a Pasteur pipette;
(6) repeating the previous step (5) three times;
(7) aliquoting a Pass solution into the flask by a 10 mL pipette, and maintaining the flask in an incubator for about 3 min;
(8) aliquoting a Neutro solution into the flask by a 10 mL pipette;
(9) collecting cells in a 15 mL tube;
(10) washing the flask with BSM;
(11) aliquoting a given amount of the cells into BCB, using a yellow tip, followed by viable cell counting;
(12) inoculating the cells into a T75 flask after viable cell counting; and
(13) mixing the remaining cells and a cell stock solution in a Cryo tube and cryopreserving the mixture; and
BRM-P2 Medium Change Kit of the osteoblast medium change and subculture process includes:
(1) taking a vinyl-wrapped BRM-P2 Medium Change Kit out of Bone RM Kit, spraying the Kit with a sufficient amount of a 70% alcohol, and transferring the Kit to a clean bench;
(2) removing a culture medium from a flask using a Pasteur pipette;
(3) aliquoting BPM into a 50 mL tube by a 25 mL pipette; and
(4) re-aliquoting a given amount of BPM from the aliquoted tube into the flask; and
BRM-P2 Subculture Kit of the osteoblast medium change and subculture process includes:
(1) taking a vinyl-wrapped BRM-P2 Subculture Kit out of Bone RM Kit, spraying the Kit with a sufficient amount of a 70% alcohol, and transferring the Kit to a clean bench;
(2) pooling and mixing the culture medium of a T75 flask in a 50 mL tube;
(3) sampling a portion of the culture medium in a 50 mL tube and an E-tube;
(4) removing the remaining culture medium by a Pasteur pipette;
(5) aliquoting BSB-II into the flask, shaking to wash the flask, and removing a supernatant by a Pasteur pipette;
(6) repeating the previous step (5) three times;
(7) aliquoting a Pass solution into the flask by a 10 mL pipette, and maintaining the flask in an incubator for about 3 to 5 min;
(8) aliquoting a Neutro solution into the flask by a 10 mL pipette;
(9) collecting cells in a 15 mL tube;
(10) washing the flask with BSM under gentle shaking;
(11) aliquoting a given amount of the cells into BCB, using a yellow tip, followed by cell counting;
(12) inoculating the cells into a T150 flask after cell counting; and
(13) mixing the remaining cells and a cell stock solution in a Cryo tube and cryopreserving the mixture; and
BRM-P3 Medium Change Kit of the osteoblast medium change and subculture process includes:
(1) taking a vinyl-wrapped BRM-P2 Medium Change Kit out of Bone RM Kit, spraying the Kit with a sufficient amount of a 70% alcohol, and transferring the Kit to a clean bench;
(2) removing the culture medium from the flask using a Pasteur pipette;
(3) aliquoting BPM into a 50 mL tube by a 25 mL pipette; and
(4) re-aliquoting a given amount of BPM from the aliquoted tube into the flask.

45. The method according to claim 41, wherein BRM-Collection Kit of the osteoblast therapy product preparation process includes:
(1) taking a vinyl-wrapped BRM-Collection Kit out of Bone RM Kit, spraying the Kit with a sufficient amount of a 70% alcohol, and transferring the Kit to a clean bench;
(2) sampling a portion of the culture medium in a 50 mL tube using a 25 mL pipette, 3 days prior to delivery of a finished product;
(3) on delivery day of the product, pooling and mixing the culture medium of the T150 flask in a 50 mL tube;
(4) sampling a portion of the culture medium in a 50 mL tube and an E-tube;
(5) removing the remaining culture medium in the flask, using a Pasteur pipette;
(6) aliquoting BSB-II into the flask by a 25 mL pipette, followed by washing;
(7) repeating the previous step (6) three times;
(8) aliquoting a Pass solution into the flask by a 10 mL pipette, and maintaining the flask in an incubator for about 4 to 5 min;
(9) aliquoting a Neutro solution into the flask by a 10 mL pipette;
(10) placing cell strainers over two 50 mL tubes, respectively;
(11) aliquoting the Pass solution-treated suspensions in the flasks into the tubes over which the cell strainers were placed, using a 25 mL pipette;
(12) aliquoting the cells into BCB using a yellow tip, and performing cell count a total of three times;
(13) aliquoting the BCM into a 1 mL vial, and mixing the cells and BCM, using a filter blue tip; and
(14) assembling a rubber cap and an aluminum cap into the 1 mL vial; and
BRM-Delivery Kit & BRM-Medium Packaging Kit of the osteoblast therapy product preparation process includes:
(1) unwrapping an outer paper packaging of BRM-Delivery Kit and BRM-Medium Packaging Kit contained in Bone RM Kit;
(2) transferring a styrofoam box with an inner packaging only to a surgical room, while leaving the removed outer packaging; and
(3) taking vials No. 1 and No. 4 out of the box transferred into the surgical room, and mixing the vials and BRM-Implantation Kit for being ready to use; and
BRM-Implantation Kit of the osteoblast therapy product preparation process includes:
(1) using a mixing tip inside BRM-Implantation Kit, mixing a medium and a substrate for use in admixture with the osteoblast therapy product in a device 2 set; and
(2) confirming full dissolution of the mixed substrate and medium composition, attaching a needle to the syringe, and injecting the composition into the bone defect site of the patient.

46. An aseptic/sterile medical kit for storage of umbilical cord blood comprising an umbilical cord blood storage kit (30) in a configuration that each process can be performed according to the functionally-specialized kit sets for each step, via division of the overall processes into the corresponding steps for isolation, culture, collection and storage of cells, and transplantation of the desired cells into the target sites of the body, wherein the umbilical cord blood storage kit includes an umbilical cord blood collection kit (31), a hematopoietic stem cell isolation kit (32), and a hematopoietic stem cell cryopreservation kit (33),
wherein the umbilical cord blood collection kit (31) includes a blood collection bag for collection of large amounts of blood, a file case for delivery of the blood collection bag, a zipper bag for storage of the blood collection bag until transport of the bag, and a sticker attached to a surface of the file case,
wherein the hematopoietic stem cell isolation kit (32) includes a processing bag for concentration of hematopoietic stem cells after removal of red blood cells, a syringe used upon sample extraction intended for quality control, a cell strainer which is placed over a centrifuge tube and separates cells from the solution by passing the resulting suspension of cells and a culture medium through it, a tube for centrifugation to wash cells, latex gloves for conducting aseptic processing operations, a CCZ vial to contain cells for storage of hematopoietic stem cells, an aluminum seal for stopping the vial, a blue tip used to take a solution, and an E-tube used upon mixing of trypan blue and the cell-containing medium,
wherein the hematopoietic stem cell cryopreservation kit (33) includes a freezing bag for freezing the hematopoietic stem cells, a cryowrap for protecting the freezing bag, and a canister for keeping the freezing bag and the cryowrap.

47. The umbilical cord blood aseptic/sterile medical kit according to claim 46, wherein the umbilical cord blood storage kit (30) further includes a medium kit (34) for isolation and cryopreservation of the hematopoietic stem cells and a medium kit (35) for isolation and cryopreservation of the hematopoietic stem cells.

48. The umbilical cord blood aseptic/sterile medical kit according to claim 46, a method of using the umbilical cord blood medical kit, for storage of umbilical cord blood by umbilical cord blood collection; hematopoietic stem cell isolation; and hematopoietic stem cell cryopreservation.

49. The method according to claim 48, wherein the umbilical cord blood collection process includes:
(1) transporting a file case of USC RM-Transport Kit contained in USC RM Kit to a surgical room;
(2) opening a zipper bag of the transported file case, and collecting umbilical cord blood using a sterile blood collection bag; and
(3) placing the collected blood in the file case of USC RM-Transport Kit and transferring the blood to a processing room.

50. The method according to claim 48, wherein the hematopoietic stem cell isolation process includes:
(1) withdrawing a given amount of blood from the blood collection bag, using a 10 mL syringe, and aliquoting the blood into an EPP tube;
(2) adding a given amount of a UES solution to the tube, using a 30 mL syringe, followed by stirring and allowing to stand at room temperature;
(3) after allowing to stand for a certain period of time, separating a primary plasma layer into the processing bag in a semi-automatic plasma separator;
(4) repeating the previous steps (2) and (3);
(5) attaching the processing bag to a bucket, followed by weighing and centrifugation;
(6) detaching the processing bag from the bucket after centrifugation;
(7) mounting the processing bag to an automatic plasma separator;
(8) aliquoting the plasma into a 15 mL tube, using the automatic plasma separator;
(9) placing the remaining plasma into a 50 mL tube, using the automatic plasma separator; and
(10) withdrawing the plasma from the 50 mL tube using a 10 mL syringe, and aliquoting the plasma into a vacumtainer.

51. The method according to claim 48, wherein the hematopoietic stem cell cryopreservation process includes:
(1) suspending a processing bag, withdrawing the contents from the suspended processing bag by a 10 mL syringe and aliquoting the contents into an EPP;
(2) withdrawing a cell stock solution by a 10 mL syringe, and adding the solution to the processing bag suspended in ice-cold water, followed by intimate mixing;
(3) sterilizing an outer surface of the processing bag with a 70% alcohol;
(4) connecting a freezing bag to the processing bag, thereby receiving a concentrated layer of nucleated cells and removing air bubbles;
(5) placing the freezing bag in a double-wrapped bag, followed by compression packaging and sterilization; and
(6) inserting the freezing bag packaged in a double-wrapped bag into the canister.
